SEQUENCE LISTING (1 sequence)

SEQ ID NO: 1 - protein, 50 aa, Artificial Sequence
  pUR4
CKDQSPLAGESGETEYITEVYGNQQNPVDIDKKLPNETGFSGNMVETEDT

(19) United States
(12) Patent Application Publication
(10) Pub. No.: US 2019/0216889 A1
(43) Pub. Date: Jul. 18, 2019

(54) METHODS AND COMPOSITIONS FOR TREATMENT OF FIBROSIS

(71) Applicant: Children's Hospital Medical Center, Cincinnati, OH (US)

(72) Inventors: Burns C. Blaxall, Cincinnati, OH (US); Prasad Devarajan, Cincinnati, OH (US); Stephanie R. Davis-Rodriguez, Villa Hills, KY (US)

(21) Appl. No.: 16/249,096

(22) Filed: Jan. 16, 2019

Related U.S. Application Data
(60) Provisional application No. 62/713,758, filed on Aug. 2, 2018.
(60) Provisional application No. 62/618,191, filed on Jan. 17, 2018.

(51) Int. Cl. A61K 38/16; A61P 13/12

(52) U.S. Cl. CPC A61K 38/164; A61P 13/12

(57) ABSTRACT

Disclosed herein are methods of treating fibrosis in an individual in need thereof. The disclosed methods employ a peptide (“pUR4”) in an amount effective to reverse, attenuate, or prevent fibrosis in an individual in need thereof. The disclosed methods and compositions may be used for treatment of fibrosis, including liver fibrosis, pulmonary fibrosis, cardiac fibrosis, kidney fibrosis, skin fibrosis, ischemia-induced fibrosis, periglomerular fibrosis, fibrosis resulting from an injury, fibrosis resulting from an obstructive event, fibrosis resulting from a nephrotoxic event, or combinations thereof.

### CROSS-REFERENCE TO RELATED APPLICATIONS

This application claims priority to and benefit of U.S. Provisional Application Ser. No. 62/618,191, to Blaxall et al., entitled “Targeting fibronectin polymerization in pathologic kidney injury and fibrosis,” filed Jan. 17, 2018 and U.S. Provisional Application Ser. No. 62/713,758, to Blaxall et al., entitled “Targeting fibronectin polymerization in pathologic kidney injury and fibrosis,” filed Aug. 2, 2018, the contents of which are incorporated in their entirety for all purposes.

### BACKGROUND

Fibrosis is a common feature of chronic kidney disease (CKD), but no therapeutics exist to effectively target its progression. Fibrosis is a key component of pathologic tissue remodeling in many human disease processes, including chronic kidney disease (CKD). The 2018 U.S. Renal Data System estimates ˜14.8% of the adult population has CKD from various etiologies.¹ The Center for Disease Control estimates a striking 54% lifetime risk of developing CKD for anyone over the age of 30.2 While several therapeutics may slow the development of CKD,^{3,4} there are currently no therapeutic approaches to halt its progression, particularly with regard to kidney fibrosis. Little is understood about the role of fibrosis in disease states, and there are essentially no FDA-approved therapies that effectively target pathologic fibrosis. Upon tissue injury, activated myofibroblasts promote extracellular matrix deposition and tissue remodeling. Fibronectin is a blood and extracellular matrix glycoprotein that is locally produced in excess quantities during this injury response. Fibronectin polymerization regulates the assembly of other extracellular matrix proteins and promotes cell adhesion, growth, migration, and contractility.

### BRIEF SUMMARY

Disclosed herein are methods of treating fibrosis in an individual in need thereof. The disclosed methods employ a peptide (“pUR4”) in an amount effective to reverse, attenuate, or prevent fibrosis in an individual in need thereof. The disclosed methods and compositions may be used for treatment of fibrosis, including liver fibrosis, pulmonary fibrosis, cardiac fibrosis, kidney fibrosis, skin fibrosis, ischemia-induced fibrosis, periglomerular fibrosis, fibrosis resulting from an injury, fibrosis resulting from an obstructive event, fibrosis resulting from a nephrotoxic event, or combinations thereof.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A-1C. Renal injury markers are decreased by pUR4 treatment following unilateral ischemia/reperfusion (UIR). 1A) Experimental timeline for UIR and peptide treatment. 1B) qPCR analysis of kidney lysates shows an increase in both lipocalin-2 and Havcr1 transcripts in injured kidneys (Inj) versus the uninjured contralateral kidney (Con) 14 days after UIR when mice are treated with PBS or III-11C (control peptide; 25 mg/kg); their expression is nearly abolished with pUR4 treatment (25 mg/kg). n=5-6 per group, *p<0.01. 1C) Immunofluorescent staining of kidneys (n=5/group) demonstrates an increase in protein expression of both NGAL (lipocalin-2) and Tim-1/Kim-1 (Havcr1) following UIR, which is decreased with pUR4 treatment. Images were obtained with a 40× objective (representative images for each group are shown), scale bar=50 μm.

FIG. 2A-2B. pUR4 attenuates immune cell infiltration and fibrosis post-UIR. 2A) Hematoxylin and eosin staining shows an expected deterioration in tubule morphology and an increase in cell infiltration following injury (upper panel; see inset), with a corresponding increase in CD45+ immunostaining (lower panel). pUR4 treatment abrogates these injury-induced observations. Kidney weight-to-body weight ratio was partially restored in pUR4-treated mice. n=8 (sham) or n=11 (peptide treatment); *p<0.0001, vs. sham; +p<0.01, III-11C vs. pUR4. Images were obtained with a 10×, 40× (H&E) or 20× (CD45) objective; scale bars=100 μm. 2B) Kidneys stained with picrosirius red show a significant increase in staining, as seen with brightfield imaging (upper panel and quantitation) and polarized light (middle panel), which is decreased with pUR4 treatment. Second harmonic generation, which specifically detects mature fibrillar collagen, also demonstrates alleviation of fibrotic collagen deposition in animals treated with pUR4. n=8 (sham) or n=11 (peptide treatment); *p<0.001 vs. sham; +p<0.001, III-11C vs. pUR4. Images were obtained with a 10× (picrosirius red) or 16× (SHG) objective; scale bars=100 μm.

FIG. 3. Inhibition of fibronectin polymerization post-UIR reduces expression of proteins associated with fibrosis and cellular activation. Immunostaining illustrates an expansion of the fibronectin and collagen I network in kidneys two weeks following UIR; treatment with pUR4 reduces this expansion and maintains more normal organization. Populations of alpha-smooth muscle actin (α-SMA)- and vimentin-positive cells are increased following injury, which is abrogated with pUR4 treatment. Note not only the change in signal intensity, but also expansion of this signal into interstitial compartments following injury. n=5/group; representative images of each group are shown. All images were obtained with a 20× objective; scale bars=100 μm.

FIG. 4. Labeled pUR4 and III-11C are evident in kidney tissue following intraperitoneal injection. Kidneys from three different animals were imaged 12 hours after injection with either PBS, III-11C-750 or pUR4-750. For histological examination, a different set of animals were injected with both III-11C-750 and pUR4-650. Twelve hours after the injections, both peptides are detected throughout the various subcompartments of the kidney. Scale bar=100 μm

FIG. 5. Schematic of experimental design. Thirty (30) minutes of unilateral renal ischemia is performed on the left kidney, while the contralateral right kidney remains untouched (Day 0). Daily injections of PBS, III-11C (25 mg/kg) or pUR4 (25 mg/kg) begin on Day 7 and continue for 7 days, at which time animals are sacrificed and tissues collected. This is an acute kidney injury-induced chronic kidney disease model, where injury results in tissue fibrosis, immune cell infiltration, immune and mesenchymal cell activation, and epithelial tubule damage. When animals are treated with pUR4 starting 7 days after surgery, however, these injury responses are blunted. This model serves as a basis for studying the progression of chronic kidney disease, as well as investigation of how fibronectin polymerization affects the injury response.

FIG. 6A-6B. Histological data for Sham, UIR+III-11C, and UIR+pUR4, and % of glomereruli surrounded by Vimentin and number of glomeruli for each.

### DETAILED DESCRIPTION

Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, or within 5-fold, or within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example, a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, or at least 10, 11, 12, 13, 14, 15, 16, 17, 18, 19, 20, 21, 22, 23, 24, or 25 contiguous nucleotides, or the full-length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Fibrosis is a hallmark of chronic kidney disease (CKD). Upon injury, activation of tissue fibroblasts leads to increased extracellular matrix (ECM) deposition and adverse tissue remodeling. Fibronectin (FN) polymerization is a requisite step in the pathogenesis of fibrosis; it nucleates collagen deposition, maturation, and matrix organization. Disclosed herein is a small therapeutic peptide, referred to herein as “pUR4,” for the specific inhibition of FN polymerization. Acute kidney injury (AM) of various etiologies, including severe dehydration, contrast imaging, invasive surgical procedures, renal ischemia, etc, often appears to resolve in the short term. However, a growing body of evidence suggests a strong likelihood for AM to progress to CKD.^{5,6} Upon tissue injury, activated inflammatory cells and fibroblasts contribute to extracellular matrix accumulation and tissue remodeling, possibly to compensate for decreased organ function and to assist with the healing response. However, persistent, chronic activation of the fibrotic response leads to organ dysfunction and failure. Mechanistic insights regarding the transition from adaptive to pathologic injury responses remain elusive, particularly in the context of CKD.

Fibronectin is a matrix glycoprotein essential for embryonic development and normal tissue function.⁷ It is a component of both specialized basement membranes and interstitial matrix, and is secreted by a number of cell types including smooth muscle cells (SMC) and fibroblasts, particularly when activated.⁸ Polymerization of secreted fibronectin is a requisite step preceding the deposition and maturation of other extracellular matrix proteins (including collagen) and affects a variety of cellular processes.⁹⁻¹¹ Fibronectin is key among the extracellular matrix proteins locally secreted by pathologically-activated cells (largely myofibroblasts) during early stages of fibrotic tissue remodeling. Since fibronectin polymerization is critical for the deposition and maturation of numerous fibrotic ECM components, its persistent expression frequently correlates with pathologic fibrosis. Although fibronectin has been considered a possible therapeutic target, the essential functions of fibronectin in tissue development and normal cell behavior have made it challenging to develop therapeutics that specifically interfere with its maladaptive role in chronic tissue remodeling.

A recombinant peptide named pUR4 has been reported in vitro to inhibit fibronectin polymerization, but not its production or secretion.^{12,13} Research in mouse models has shown that localized pUR4 treatment attenuates excess ECM deposition in vascular remodeling¹⁴ and liver fibrosis¹⁵. We recently reported that one week of systemic daily pUR4 administration improves cardiac function and decreases pathologic fibrosis and cardiac remodeling up to four weeks after myocardial ischemia/reperfusion (I/R) injury.¹⁶ Specifically, pUR4 treatment reduced proliferation of activated cardiac myofibroblasts, attenuated the deposition of ECM proteins and reduced neutrophil infiltration, all of which contributed to the amelioration of cardiac dysfunction following I/R.

Disclosed herein is a method of treating fibrosis in an individual in need thereof. The method may comprise the step of administering to an individual suspected of having, having, or likely to develop fibrosis, an amount of a peptide having at least 95%, 96%, 97%, 98%, 99%, or 100% sequence identity to the sequence CKDQSPLAGESGETEYITEVYGNQQNPVDIDKKLPNETGFSGNMVETEDT (SEQ ID NO: 1) (“pUR4”). The composition may be administered in an amount effective to reverse, attenuate, or prevent fibrosis in said individual.

In one aspect, the individual may have fibrosis selected from liver fibrosis, pulmonary fibrosis, cardiac fibrosis, kidney fibrosis, skin fibrosis, fibrosis resulting from an injury, including injury from an ischemic event, an obstructive event, a nephrotoxic event, or a combination thereof. In one aspect, the administration step may be repeated until normalization or improvement of kidney function as determined by measurement of BUN, creatinine, or combinations thereof, wherein a normalization or improvement of BUN and/or creatinine values indicates a normalization or improvement of kidney function. In one aspect, an effective amount may be an amount sufficient to decrease kidney injury markers, attenuate fibronectin and collagen deposition, decrease inflammatory cell infiltration, reduce markers of fibroblast activation. In one aspect, the effective amount may be an amount sufficient to inhibit fibronectin polymerization, as determined by decreased expression of kidney injury markers following said administration, wherein said kidney injury markers are lipocalin-2 (NGAL), Havcr1 (Kim1), or combinations thereof. In one aspect, the effective amount may be determined by decreased expression of fibroblast activation and inflammation markers following said administration, wherein said markers are selected from alpha-SMA, vimentin, CD45, uromodulin, E-cadherin, or combinations thereof.

The disclosed peptides may be administered systemically, for example intravenously or intraperitoneally. In one aspect, the disclosed peptides, for example pUR4, may be administered pen-surgically, such as wherein said administration is directly (locally) to the site of fibrosis. In one aspect, the peptide may be administered via a nanocage, a nanoparticle, or a combination thereof.

In one aspect, the administration step may comprise a first administration comprising administering a bolus dose, and a second administration comprising a second dose that is less than said bolus dose. In certain aspects, the administration step may comprise administering a dose of about 25 mg/kg, or from about 5 mg/kg to about 100 mg/kg, or from about 10 mg/kg to about 75 mg/kg, or from about 20 mg/kg to about 50 mg/kg.

In one aspect, a method of treating ischemia-induced fibrosis, for example, periglomerular fibrosis is disclosed. The method may comprise the step of administering pUR4, or a peptide having at least 95%, 96%, 97%, 98%, 99%, or 100% sequence identity to (SEQ ID NO: 1) CKDQSPLAGESGETEYITEVYGNQQNPVDIDKKLPNETGFSGNMVETEDT (“pUR4”).

Polysaccharide polymers such as chitosan are also well known as gel forming medicinal agents. Chitosan is recognized to have wound healing properties. For example, U.S. Pat. No. 5,836,970 discloses chitosan and alginate wound dressings that may be prepared as fibers, powders, films, foams, or water-swellable hydrocolloids. U.S. Pat. No. 5,599,916 discloses a water-swellable, water-insoluble chitosan salt that can be used in wound dressings, and U.S. Pat. No. 6,444,797 discloses a chitosan microflake that can be used as a wound dressing or skin coating.

In one aspect, a method of treating fibrosis in an individual in need thereof is disclosed, wherein the fibrosis results from a fibrotic stimulus selected from ischemia, inflammation, diet, or a combination thereof, comprising administering a composition comprising administering a peptide having at least 95%, 96%, 97%, 98%, 99%, or 100% sequence identity to the sequence (SEQ ID NO: 1) CKDQSPLAGESGETEYITEVYGNQQNPVDIDKKLPNETGFSGNMVETEDT (“pUR4”) in an amount effective to reverse, attenuate, or prevent fibrosis in the individual.

Pharmaceutical Compositions

In one aspect, a composition comprising a sequence having at least 95% sequence identity to (SEQ ID NO: 1) CKDQSPLAGESGETEYITEVYGNQQNPVDIDKKLPNETGFSGNMVETEDT (“pUR4”) is disclosed. The composition may be in the form of a gel, for example, in a form suited for being applied directly to a biological surface, for example, during a surgical procedure. Exemplary gels include those disclosed in U.S. Pat. No. 8,865,680B2, “Surgical Gel System,” U.S. Pat. No. 7,083,806B2, “Wound gels,” and US20100291055 A1, “Surgical hydrogels.” For example, the disclosed peptide may be incorporated into a polymer solution or gel that may then be applied to target areas. Such gels may be used to coat surgically exposed tissues before closing the surgical site. Polymers having the disclosed peptides may be added to the patient in situ, in a solution and then chemically reacted to form covalent cross-links so as to create a polymer network. For example, SprayGelTM is a PEG-based material that forms an adhesion barrier when applied to tissue, which may be used in conjunction with the disclosed peptides. The gel or hydrogel may be made by combining aqueous solutions of two or more polymers that cross-link to foam a polymer network when mixed. As cross-linking occurs, the resulting polymer network forms a gel or hydrogel in aqueous solution. The hydrogel can be formed in situ, for example, by spraying, squirting or pouring the polymer solutions onto the target area. Alternatively, the gel or hydrogel can be pre-formed, then applied to the target area. In another embodiment, the gel or hydrogel can be formed when a wound dressing incorporating the polymer components is moistened. A further example includes SURGIFLO™ Haemostatic Matrix, which is a sterile, absorbable porcine gelatin paste intended for haemostatic use by applying to a bleeding surface. The gelatin paste is supplied in a pre-filled syringe to be mixed with 2 ml of additional liquid (sterile saline solution or thrombin) and may be combined with the peptides of the instant disclosure.

In one aspect, active agents provided herein may be administered in an dosage form selected from intravenous or subcutaneous unit dosage form, oral, parenteral, intravenous, and subcutaneous. In some embodiments, active agents provided herein may be formulated into liquid preparations for, e.g., oral administration. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, active agents provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricant such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

In some embodiments, each tablet or capsule contains from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some embodiments, tablets or capsules are provided in a range of dosages to permit divided dosages to be administered. A dosage appropriate to the patient and the number of doses to be administered daily may thus be conveniently selected. In certain embodiments two or more of the therapeutic agents may be incorporated to be administered into a single tablet or other dosage form (e.g., in a combination therapy); however, in other embodiments the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragamayth, or alginic acid or salts thereof.

Binders may be used to form a hard tablet. Binders include materials from natural products such as acacia, tragamayth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy.

The active ingredients may be prepared for pulmonary delivery in particulate form with an average particle size of from 0.1 um or less to 10 um or more, for example, from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used. Liposomes, microcapsules, microspheres, inclusion complexes, and other types of carriers may also be employed.

Pharmaceutical formulations suitable for use with a nebulizer, either jet or ultrasonic, typically comprise the active agent dissolved or suspended in water at a concentration of about 0.01 or less to 100 mg or more of active agent per mL of solution, for example, from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation may also include a buffer and a simple sugar (e.g., for protein stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device generally comprise a finely divided powder containing the active ingredients suspended in a propellant with the aid of a surfactant. The propellant may include conventional propellants, such as chlorofluorocarbons, hydrochlorofluorocarbons, hydrofluorocarbons, and hydrocarbons. Example propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing active agent, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, for example, from about 5, 10, 15, 20, 25, 30, 35, 40, 45, or 50 wt. % to about 55, 60, 65, 70, 75, 80, 85, or 90 wt. % of the formulation.

In some embodiments, an active agent provided herein may be administered by intravenous, parenteral, or other injection, in the form of a pyrogen-free, parenterally acceptable aqueous solution or oleaginous suspension. Suspensions may be formulated according to methods well known in the art using suitable dispersing or wetting agents and suspending agents. The preparation of acceptable aqueous solutions with suitable pH, isotonicity, stability, and the like, is within the skill in the art. In some embodiments, a pharmaceutical composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art.

The duration of the injection may be adjusted depending upon various factors, and may comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

In some embodiments, active agents provided herein may additionally employ adjunct components conventionally found in pharmaceutical compositions in their art-established fashion and at their art-established levels. Thus, for example, the compositions may contain additional compatible pharmaceutically active materials for combination therapy or may contain materials useful in physically formulating various dosage forms, such as excipients, dyes, thickening agents, stabilizers, preservatives or antioxidants.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating the disease states described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Fibrosis is a common feature of chronic kidney disease (CKD), but no therapeutics exist to effectively target its progression. Fibronectin polymerization is a requisite step preceding deposition of other matrix proteins, including collagen. While this process is required for normal development and injury responses, excess deposition and polymerization of fibronectin correlates with the elaboration of fibrosis. pUR4 is a small peptide that inhibits polymerization of fibronectin, but not its production nor secretion. Applicant has investigated pUR4 treatment for the abrogation of ischemia/reperfusion-induced kidney fibrosis and maladaptive tissue remodeling.

Methods: Male C57B1/6 mice underwent sham surgery or had an atraumatic clamp placed on the left renal artery and vein (unilateral ischemia/reperfusion (UIR)) for 30 minutes. Starting seven days after surgery, pUR4 (25 mg/kg/d), III-11C (control; 25 mg/kg/d) or PBS were administered daily by intraperitoneal injection. Mice were sacrificed and tissues collected 14 days after surgery.

Results: Following renal UIR injury, treatment with pUR4 reduced lipocalin-2 (NGAL) and havcr-1 (Kim-1) gene and protein expression, attenuated fibrosis and pathological tissue remodeling, and decreased immune cell infiltration into the renal parenchyma.

Conclusions: Inhibition of fibronectin polymerization with pUR4 improved renal morphology and decreased fibrotic tissue remodeling in injured kidneys. Thus, pUR4 administration may be used to inhibit progression from acute kidney injury to chronic kidney disease and renal failure.

Applicant has found that pUR4 abrogates the development of fibrosis and adverse tissue remodeling observed following renal injury. In short, Applicant has found that partial recovery of kidney mass, attenuation of fibrosis and pathologic tissue remodeling, and a decrease in immune cell infiltration into the renal parenchyma can be achieved following administration of pUR4. Studies were performed in a murine model of unilateral renal ischemia/reperfusion (UIR), which is relevant to many clinical scenarios where an acute ischemic event in the kidney leads to or exacerbates chronic kidney disease.^{17,18}

Results and Discussion

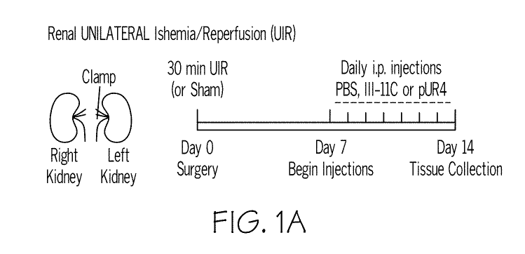

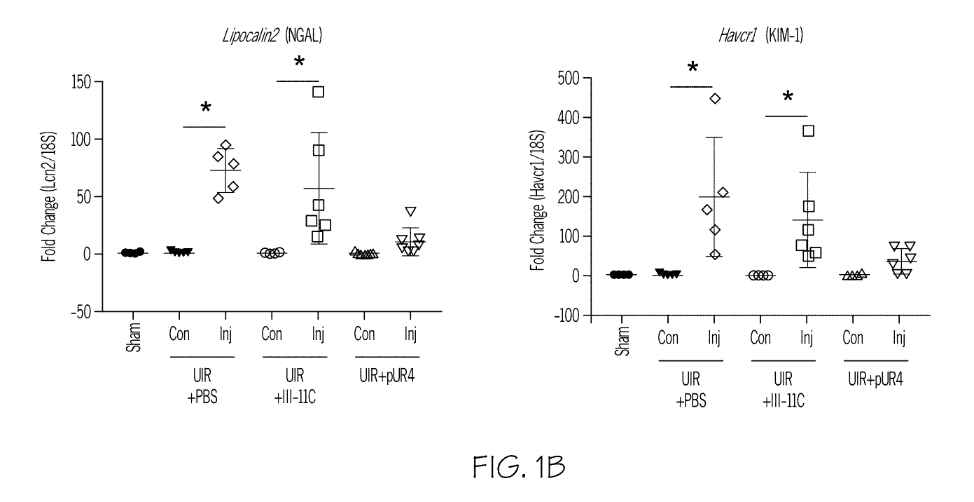

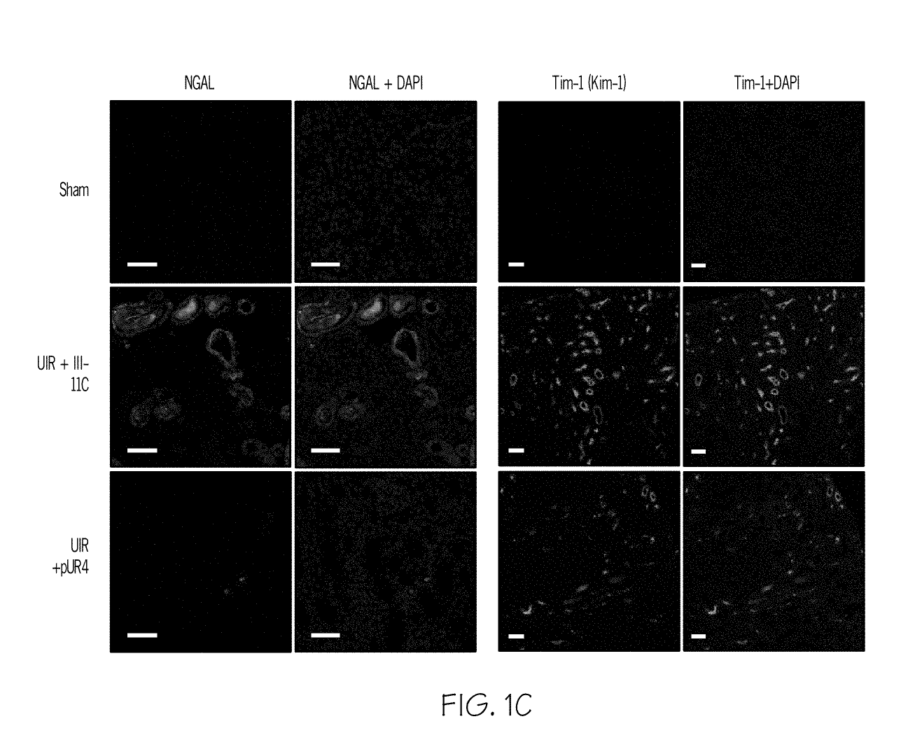

Following a dose-escalation and timing study, initiation of pUR4 treatment 7 days after injury proved to be most successful in treating pathologic fibrosis (see FIG. 1A and the Schematic). Expression of injury markers lipocalin-2 (Lcn-2/NGAL) and havcr1 (Kim1/Tim1) were significantly elevated 14 days following UIR in both PBS- and III-11C-treated mice. However, pUR4 treatment initiated 7 days post-UIR significantly reduced expression of these injury markers in injured versus contralateral kidneys (FIG. 1B). Immunostaining demonstrated an increase in NGAL and Timl (the mouse ortholog of human Kim1) protein expression 2 weeks following UIR, which were both decreased after pUR4 treatment (FIG. 1C).

While all control groups were included in each experiment, no differences were observed in animals that underwent sham surgery followed by injection with PBS, III-11C or pUR4, nor was any effect observed in contralateral (uninjured) kidneys in mice subjected to UIR when compared to sham controls. Furthermore, UIR+PBS and UIR+III-11C were not different from one another.

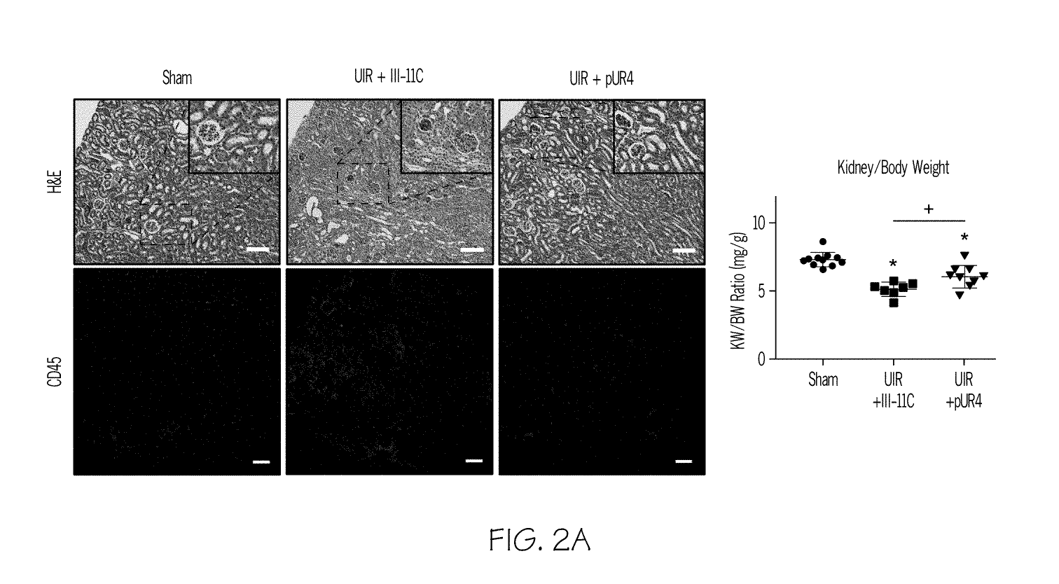

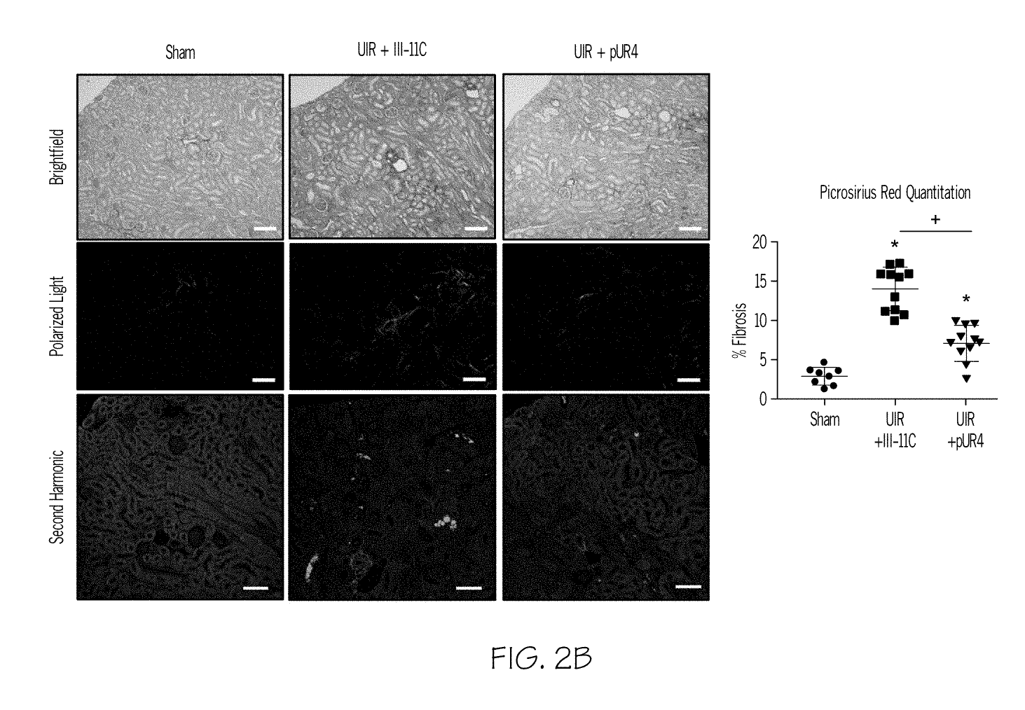

H&E staining confirmed tissue damage following ischemic injury, including tubule dropout and an increase in cellular infiltration, as seen in injured kidneys of III-11C-treated mice compared to sham (FIG. 2A, top panel). Following pUR4 treatment, a decrease in cellular infiltrate and reduced tubule damage was evident (insets in FIG. 2A). Immunostaining with CD45 confirmed an accumulation of immune cells two weeks following injury, which was abrogated with pUR4 treatment (FIG. 2A, bottom panel). Furthermore, while kidney weights were decreased two weeks after UIR, pUR4 treatment significantly recovered kidney weight, indicating a preservation of tissue composition (FIG. 2A, right panel).

Fibrosis is largely characterized by detrimental accumulation of extracellular matrix and can be assessed using picrosirius red staining of collagen fibrils. Treatment with pUR4 following UIR attenuated the deposition and maturation of collagen when compared to III-11C, as examined by both brightfield (FIG. 2B, upper panel and graph quantitation) and polarized light (middle panel) imaging of sections stained with picrosirius red. Second harmonic generation (SHG) with multi-photon imaging is an emerging method to non-histologically assess native, triple-helical fibrillar collagen;19 SHG corroborated the observation that pUR4 treatment decreased kidney fibrosis (FIG. 2B, lower panel).

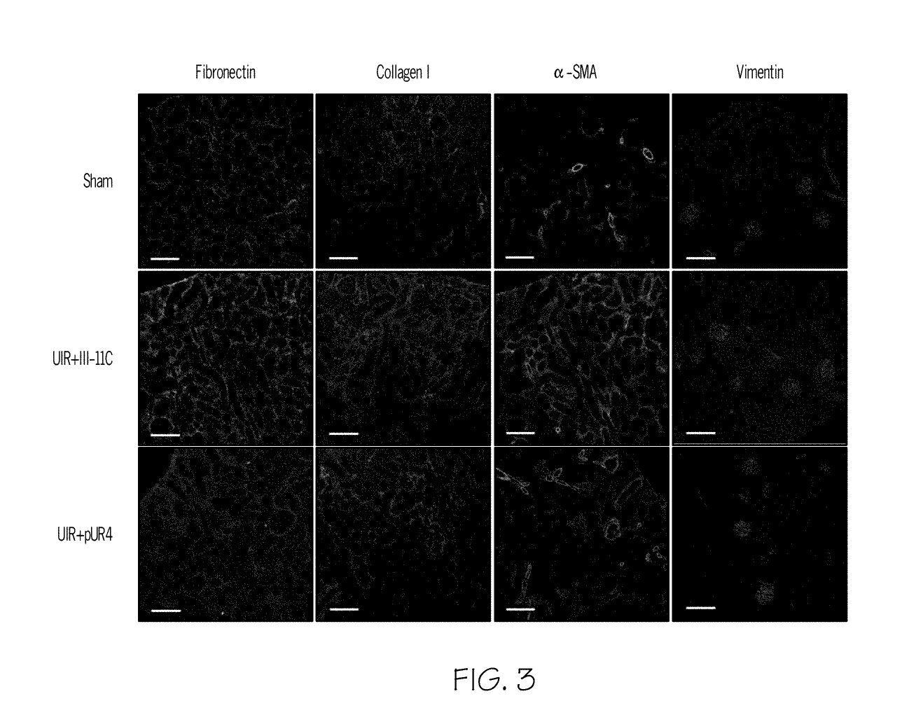

To further characterize how pUR4 affects matrix deposition in pathologic remodeling, fibronectin and collagen I were evaluated by immunofluorescence. UIR increased deposition of fibronectin and collagen I after 14 days, whereas pUR4 treatment after UIR decreased the amount of fibronectin present in kidney interstitium (FIG. 3). Attenuated polymerization of fibronectin and its incorporation into the ECM is likely responsible for the concomitant decrease observed in collagen I deposition. pUR4 treatment also diminished the injury-induced increase in alpha-smooth muscle actin (α-SMA) and vimentin expression, which are both markers of fibroblast activation observed during the initiation and progression of fibrosis (FIG. 3). In normal kidneys, α-SMA is largely expressed in smooth muscle cells around larger arteries, but a small amount is also seen surrounding glomeruli and in the tubulointerstitium. However, these α-SMA-expressing cell populations expand after injury (largely as interstitial myofibroblasts; see FIG. 3). Treatment with pUR4 reduced this expansion and overall α-SMA expression, which corroborates reported effects of pUR4 in vascular remodeling.¹⁴

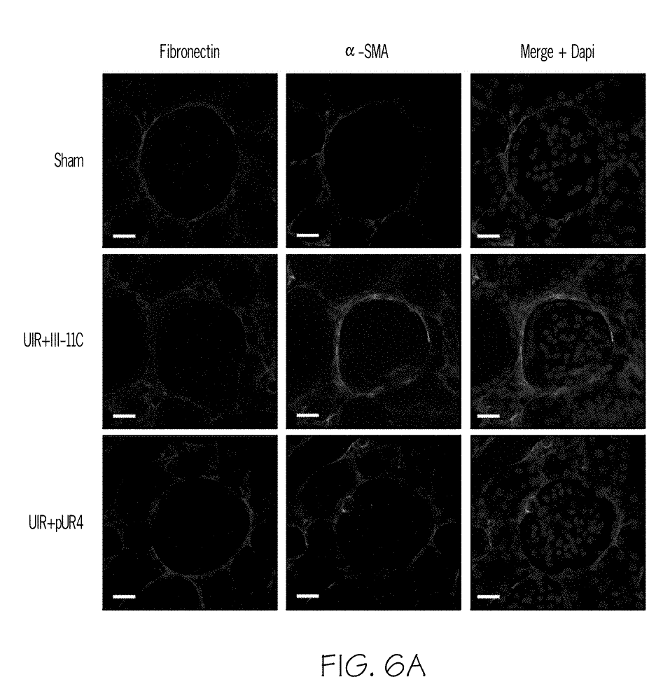

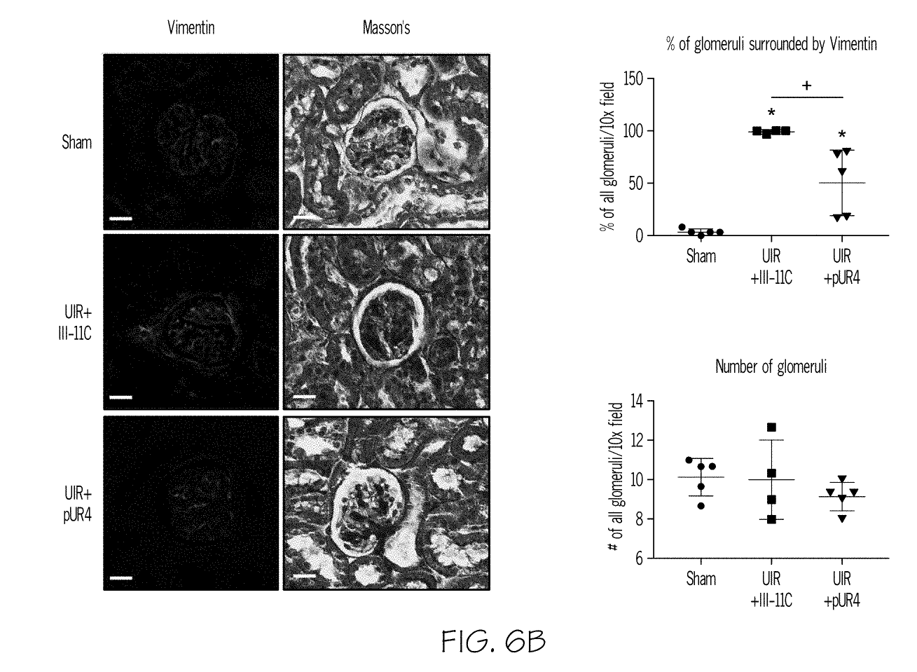

Vimentin is normally observed in podocytes within glomeruli, but it is also expressed by activated cells (largely myofibroblasts) that expand into periglomerular and tubulointerstitial spaces after injury.^{20,21} In addition to mediating α-SMA-positive cell activation, pUR4 abrogated this expansion of vimentin-positive cells outside of the glomerulus, as a decrease in interstitial vimentin was observed (FIG. 3 and FIGS. 6A and 6B). Closer observations revealed that pUR4 reduced periglomerular fibrosis, as indicated by reduced fibronectin, α-SMA and vimentin in the areas immediately surrounding glomeruli (FIGS. 6A and 6B). Collectively, decreased ECM accumulation and markers of cellular activation were observed with pUR4 treatment following UIR.

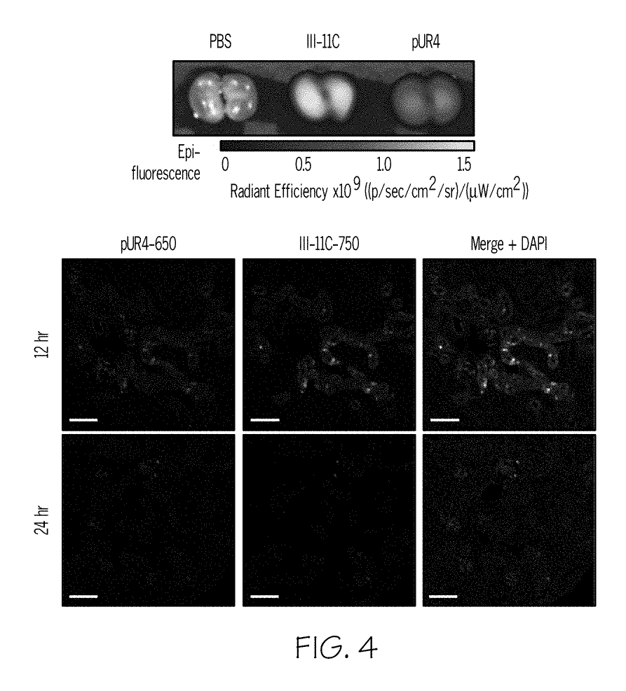

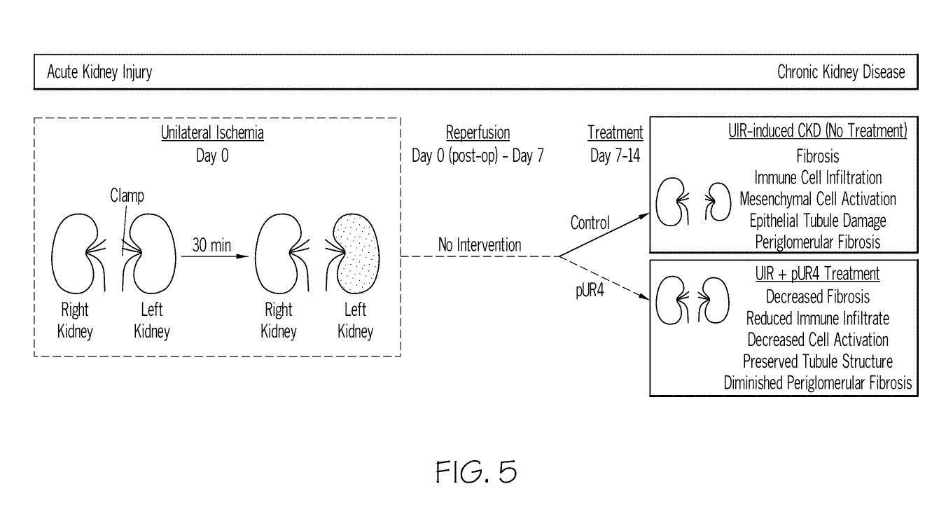

To explore possible mechanisms of action and investigate appropriate distribution of the peptides, pUR4 and III-11C were conjugated to far-red (650 nm) and/or near-infrared (750 nm) fluorophores to facilitate visualization. Whole-organ imaging demonstrated the presence of either pUR4-750 or III-11C-750 peptide in the kidneys 12 hours after i.p. injection (FIG. 4, top). In a separate experiment, animals were injected with both pUR4-650 and III-11C-750; tissues were harvested, fixed and paraffin-embedded for microscopic analysis 12 hours following delivery of the peptides. Confocal imaging confirmed that the peptides are indeed delivered throughout the various compartments within the kidney (FIG. 4 panels).

Overall, targeting fibronectin polymerization during the transition from acute injury to maladaptive fibrotic remodeling was found to attenuate the progression of pathologic fibrosis and organ failure.¹⁴⁻¹⁶ Indeed, Applicant's theory finds support in the recent literature, pUR4 having recently been shown to attenuate fibrosis in an obstructive model of kidney injury, unilateral ureteral obstruction (UUO).²² The study reported herein strongly advocates for pUR4 as a novel therapeutic approach to ameliorating kidney fibrosis, a common and progressive condition for which no FDA-approved therapies are currently available.

Materials and Methods

Animals

All procedures were approved by and performed according to the Department of Laboratory Animal Medicine and the Institutional Animal Care and Use Committee at the Cincinnati Children's Hospital Medical Center. Wild type C57BL6/J 9-10-week-old male mice (Jackson Laboratories) were used for these experiments.

Unilateral Ischemia/Reperfusion and pUR4 Administration

Mice were allowed 1-2 weeks of acclimation upon arrival to the animal facility, after which they underwent unilateral ischemia/reperfusion (UIR) according to Le Clef et al., 2016.17 Briefly, mice were anesthetized with 3% isoflurane for surgical prep (abdominal hair removal, animal identification, etc.), and maintained at 1.5% isoflurane on a 37° C. heating pad throughout the procedure. Following surgical exposure of the ventral abdomen, an atraumatic clamp was placed on the left renal artery and vein for 30 minutes; verification of color change following reperfusion was performed by the surgeon before mice were sutured and glued (outer suture reinforcement), given buprenorphine and subcutaneous saline and returned to their cages. For each animal, the contralateral (right) kidney and renal blood vessels were visualized, but remained untouched. Sham animals underwent an identical surgical procedure, except that the left and right renal arteries and veins were exposed, but were not clamped. Seven (7) days after surgery, mice were weighed and randomly assigned to daily intraperitoneal (i.p.) injections of PBS, III-11C (control peptide; 25 mg/kg), or pUR4 (25 mg/kg).

Tissue Collection

14 days after UIR (7 days after the onset of daily peptide injections), mice were given 100U heparin and anesthetized with isoflurane; blood was collected and animals were perfused with cold PBS through the aortic arch. Kidneys were excised (and the capsule membrane carefully removed), weighed, and then bisected lengthwise; one half was placed in cold 4% paraformaledhyde overnight followed by paraffin embedding, the other half cut into ˜1 mm3 pieces and flash-frozen for RNA and protein isolation.

Histology and Immunostaining

Fixed kidneys were paraffin embedded, sectioned at 8 μm and mounted onto glass slides. Prior to staining, the sections were deparaffinized in xylene and rehydrated to water through increasing dilutions of ethanol (100%, 95%, 70%, and then 50%). For picrosirius red staining, slides were incubated for 1 hour in 0.1% Sirius Red (in 1.3% aqueous picric acid), washed twice in acidified water (0.5% acetic acid), cleared in ethanol, dehydrated in xylene and mounted with Permount. Images were taken at 10× and 40× (Olympus BX-51 with a DS-Ril camera), and the percentage of red staining was quantified using Image J software (NIH) by two observers blinded to experimental conditions.

For immunofluorescent staining, deparaffinized and rehydrated sections were microwaved for 20 minutes in a sodium citrate antigen retrieval solution (Vector H-3300), and then permeabilized in 0.06% triton-X in TBS. Sections were blocked in 10% goat or donkey serum (depending on the secondary antibody) in TBS for 2 hours at room temperature, and then incubated with primary antibody in 1% serum/TBS overnight at 4° C. Antibodies used were as follows: lipocalin-2/NGAL (1:100; JM-3819, MBL Intl Corp), Tim1/Kim1 (1:100; AF1817, R&D Systems), CD45 (1:100; AF114, R&D Systems), fibronectin (1:100; ab2413, Abcam), collagen I (1:100; ab21286, Abcam), vimentin (1:100; ab45939, Abcam), alpha-smooth muscle actin (1:500; A5228, Sigma). Slides were then washed three times in TBST for 5 min each, and incubated with secondary antibodies for 45 minutes at room temperature. In these experiments the following secondary antibodies were used, all from Jackson ImmunoResearch: donkey anti-rabbit 488 (711-545-152), donkey anti-goat 488 (705-545-147), donkey anti-mouse 647 (715-605-150) and donkey anti-rabbit 647 (711-605-152). Following incubation with secondary antibodies, slides were washed with TBST twice, TBS once, and then incubated with DAPI (1:1000; #62248, ThermoScientific) for 20 minutes at room temperature, washed again in TBS, and mounted with ProLong Gold (P36934, Invitrogen/Thermo Fisher Scientific). Sections were imaged on a Nikon AIR inverted microscope by an individual blinded to experimental conditions, with objectives for 10×, 20× and/or 40×, as indicated in figure legends. For quantitation of glomerular phenotype, a blind observer used three-10× images per kidney to assess the average number of glomeruli per field, as well as the percentage of glomeruli surrounded by vimentin.

qPCR

Total RNA was extracted from ˜20 mg kidney tissue using a GeneJet RNA extraction kit, according to the manufacturers' protocol (Fisher Scientific K0732). Complementary DNA was synthesized by reverse transcription using the iScript cDNA synthesis kit (BioRad 1708841). Quantitative real-time polymerase chain reaction (qRT-PCR) was performed on a StepOne Plus Real-Time PCR machine (Applied Biosystems) using Taqman primer-probe sets for lcn-2 (Mm01324470_ml) and havcr1 (Mm00506686_ml). Samples were normalized to 18S (4352930E; AP Bio). Transcript levels were determined using comparative ΔΔCT method, and averaged within groups. mRNA expression is reported as fold change relative to sham-treated kidneys.

Second Harmonic Generation (SHG)

SHG imaging was performed on unstained paraffin-embedded sections (8 μm) using a Nikon MR multiphoton upright microscope with a coherent Chameleon II TiSapphire laser tuned to 840 nm.19 SHG signal was detected from backward emission via non-descanned detectors (NDDs) using a 16× objective.

Peptide Production and Purification

The peptides pUR4 and III-11C were synthesized as described¹⁴ using the expression vectors pQE30 and pQE12, respectively, in M15 E. coli. Recombinant His-tagged peptides were recovered using HisPur Ni-NTA spin columns, filtered to remove endotoxin, and dialyzed into PBS. Protein concentration was determined by BCA assay, and SDS-page gel electrophoresis with Coomassie Blue staining confirmed a single protein band at the appropriate molecular weight. Endotoxin levels were measured with a Limulus amebocyte lysate assay kit (Lonza 50-647U) to verify that samples contained less than 0.1 endotoxin units per mg of peptide.

Fluorescent Labeling and In Vivo Imaging System (IVIS)

Peptides were labeled with TideFluor 5WS, succinimidyl ester (TFSWS-SE; #2281, AAT Bioquest) or TideFluor 7WS, succinimidyl ester (TF7WS-SE; #2333, AAT Bioquest) according to the manufacturers' protocol. For whole-organ imaging, mice were injected i.p. with PBS, III-11C-750 (TF7WS-SE) or pUR4-750; after 12 hours, kidneys were harvested and quickly imaged on an IVIS 200 Series imaging system. For tissue analysis, mice were injected with both III-11C-750 and pUR4-650 (TFSWS-SE), or PBS alone. At 12 hours post-injection, mice were perfused with cold PBS, and kidneys were collected, fixed in 4% paraformaldehyde and paraffin embedded. 8 μm sections were mounted on glass slides, cleared with xylene, rehydrated in ethanol (2 changes of 100%, 95%, 70%, and then 50%), rinsed in TBS, stained with DAPI and then mounted in ProLong Gold. Images were obtained on a Nikon MR inverted confocal microscope.

Statistical Analysis

Data were analyzed by one-way ANOVAs using GraphPad Prism 7 software, and are presented as average +/− standard deviation. Results were considered statistically significant when p<0.05.

### REFERENCES

1. System, USRD: 2018 USRDS Annual Report: Epidemiology of kidney disease in the United States. National Institutes of Health, National Institute of Diabetes and Digestive and Kidney Diseases, Bethesda, MD, 2018.

2. Hoerger, T J, Simpson, S A, Yarnoff, B O, Pavkov, M E, Rios Burrows, N, Saydah, S H, Williams, D E, Zhuo, X: The future burden of CKD in the United States: a simulation model for the CDC CKD Initiative. Am J Kidney Dis, 65: 403-411, 2015.

3. System, USRD: 2013 USRDS Annual Report: Atlas of CKD and ESRD. National Institutes of Health, National Institute of Diabetes and Digestive and Kidney Diseases, Bethesda, Md., 2013.

4. Foundation, NK: KDOQI clinical practice guideline for diabetes and CKD: 2012 update. Am J Kidney Dis, 60, 2012.

5. Chawla, L S, Eggers, P W, Star, R A, Kimmel, P L: Acute kidney injury and chronic kidney disease as interconnected syndromes. N Engl J Med, 371: 58-66, 2014.

6. Coca, S G, Singanamala, S, Parikh, C R: Chronic kidney disease after acute kidney injury: a systematic review and meta-analysis. Kidney Int, 81: 442-448, 2012.

7. George, E L, Georges-Labouesse, E N, Patel-King, R S, Rayburn, H, Hynes, R O: Defects in mesoderm, neural tube and vascular development in mouse embryos lacking fibronectin. Development, 119: 1079-1091, 1993.

8. Hynes, R O: Fibronectins, New York, Springer-Verlag, 1990.

9. Hocking, D C, Sottile, J, Langenbach, K J: Stimulation of integrin-mediated cell contractility by fibronectin polymerization. J Biol Chem, 275: 10673-10682, 2000.

10. Sottile, J, Hocking, D C: Fibronectin polymerization regulates the composition and stability of extracellular matrix fibrils and cell-matrix adhesions. Mol Biol Cell, 13: 3546-3559, 2002.

11. Veiling, T, Risteli, J, Wennerberg, K, Mosher, DF, Johansson, S: Polymerization of type I and III collagens is dependent on fibronectin and enhanced by integrins alpha llbeta 1 and alpha 2beta 1. J Biol Chem, 277: 37377-37381, 2002.

12. Ensenberger, M G, Tomasini-Johansson, B R, Sottile, J, Ozeri, V, Hanski, E, Mosher, D F: Specific interactions between F1 adhesin of Streptococcus pyogenes and N-terminal modules of fibronectin. J Biol Chem, 276: 35606-35613, 2001.

13. Tomasini-Johansson, B R, Kaufman, N R, Ensenberger, M G, Ozeri, V, Hanski, E, Mosher, D F: A 49-residue peptide from adhesin F1 of Streptococcus pyogenes inhibits fibronectin matrix assembly. J Biol Chem, 276: 23430-23439, 2001.

14. Chiang, H Y, Korshunov, V A, Serour, A, Shi, F, Sottile, J: Fibronectin is an important regulator of flow-induced vascular remodeling. Arterioscler Thromb Vasc Biol, 29: 1074-1079, 2009.

15. Altrock, E, Sens, C, Wuerfel, C, Vasel, M, Kawelke, N, Dooley, S, Sottile, J, Nakchbandi, I A: Inhibition of fibronectin deposition improves experimental liver fibrosis. J Hepatol, 62: 625-633, 2015.

16. Valiente-Alandi, I, Potter, S J, Salvador, A M, Schafer, A E, Schips, T, Carrillo-Salinas, F, Gibson, A M, Nieman, M L, Perkins, C, Sargent, M A, Huo, J, Lorenz, J N, DeFalco, T, Molkentin, J D, Alcaide, P, Blaxall, B C: Inhibiting Fibronectin Attenuates Fibrosis and Improves Cardiac Function in a Model of Heart Failure. Circulation, 138: 1236-1252, 2018.

17. Le Clef, N, Verhulst, A, D'Haese, P C, Vervaet, B A: Unilateral Renal Ischemia-Reperfusion as a Robust Model for Acute to Chronic Kidney Injury in Mice. PLoS One, 11: e0152153, 2016.

18. Zager, R A, Johnson, A C, Becker, K: Acute unilateral ischemic renal injury induces progressive renal inflammation, lipid accumulation, histone modification, and “end-stage” kidney disease. Am J Physiol Renal Physiol, 301: F1334-1345, 2011.

19. Konig, K: Multiphoton microscopy in life sciences. J Microsc, 200: 83-104, 2000.

20. Li, Y, Kang, Y S, Dai, C, Kiss, L P, Wen, X, Liu, Y: Epithelial-to-mesenchymal transition is a potential pathway leading to podocyte dysfunction and proteinuria. Am J Pathol, 172: 299-308, 2008.

21. Reiser, J, Altintas, M M: Podocytes. F1000Res, 5, 2016.

22. Tomasini-Johansson, B R, Zbyszynski, P W, Toraason, I, Peters, D M, Kwon, G S: PEGylated pUR4/FUD peptide inhibitor of fibronectin fibrillogenesis decreases fibrosis in murine Unilateral Ureteral Obstruction model of kidney disease. PLoS One, 13: e0205360, 2018.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of treating fibrosis in an individual in need thereof, comprising administering to said individual of a peptide having at least 95% sequence identity to the sequence CKDQSPLAGESGETEYITEVYGNQQNPVDIDKKLPNETGFSGNMVETEDT (SEQ ID NO: 1) (“pUR4”) in an amount effective to reverse, attenuate, or prevent fibrosis in said individual.

2. The method of claim 1, wherein said fibrosis is selected from liver fibrosis, pulmonary fibrosis, cardiac fibrosis, kidney fibrosis, skin fibrosis, ischemia-induced fibrosis, periglomerular fibrosis, fibrosis resulting from an injury, fibrosis resulting from an obstructive event, fibrosis resulting from a nephrotoxic event, or a combination thereof.

3. The method of claim 1, wherein said administration step is repeated until normalization or improvement of kidney function is achieved, as determined by measurement of BUN, creatinine, or combinations thereof, wherein a normalization or improvement of BUN and/or creatinine values indicates a normalization or improvement of kidney function.

4. The method of claim 1, wherein said effective amount is an amount sufficient to effect a result selected from decreased kidney injury markers, attenuated fibronectin and collagen deposition, decreased inflammatory cell infiltration, reduced markers of fibroblast activation, or combinations thereof.

5. The method of claim 1, wherein said effective amount is an amount sufficient to inhibit fibronectin polymerization, as determined by decreased expression of kidney injury markers following said administration, wherein said kidney injury markers are lipocalin-2 (NGAL), Havcr1 (Kim1l), or combinations thereof.

6. The method of claim 1, wherein said effective amount is determined by decreased expression of fibroblast activation and inflammation markers following said administration, wherein said markers are selected from alpha-SMA, vimentin, CD45, uromodulin, E-cadherin, or combinations thereof.

7. The method of claim 1, wherein said pUR4 is administered systemically.

8. The method of claim 1, wherein said pUR4 is administered pen-surgically.

9. The method of claim 1, wherein said pUR4 is administered via a nanocage, a nanoparticle, or a combination thereof.

10. The method of claim 1, wherein said administration step comprises a first administration comprising administering a bolus dose, and a second administration comprising a second dose that is less than said bolus dose.

11. The method of claim 1, wherein said administration comprises a dose of about 25 mg/kg, or from about 5 mg/kg to about 100 mg/kg, or from about 10 mg/kg to about 75 mg/kg, or from about 20 mg/kg to about 50 mg/kg.

12. A composition comprising a sequence having at least 95% sequence identity to CKDQSPLAGESGETEYITEVYGNQQNPVDIDKKLPNETGFSGNMVETEDT (SEQ ID NO: 1) (“pUR4”), wherein said composition is a gel or hydrogel.

13. A method of treating fibrosis in an individual in need thereof, wherein said fibrosis results from a fibrotic stimulus selected from ischemia, inflammation, diet, or a combination thereof, comprising administering a composition comprising a peptide having at least 95% sequence identity to the sequence CKDQSPLAGESGETEYITEVYGNQQNPVDIDKKLPNETGFSGNMVETEDT (SEQ ID NO: 1) (“pUR4”) effective to reverse, attenuate, or prevent fibrosis in said individual.
